(19) United States
(12) Patent Application Publication
(10) Pub. No.: US 2021/0322149 A1
(43) Pub. Date: Oct. 21, 2021

(54) CORNEAL DEVICE POSITIONING SYSTEMS, DEVICES, AND METHODS

(71) Applicant: CN.USA BIOTECH HOLDINGS, INC., Houston, TX (US)

(72) Inventors: Niclas Henning ZIEGER, Pasadena, CA (US); Nicholas J. MANESIS, Escondido, CA (US); Nicolas ESGUERRA, Irvine, CA (US); Rachel Marie WONG, Costa Mesa, CA (US)

(21) Appl. No.: 16/625,497

(22) PCT Filed: Jun. 22, 2018
(86) PCT No.: PCT/US2018/038925

§ 371 Date: Dec. 20, 2019

Related U.S. Application Data
(60) Provisional application No. 62/524,201, filed on Jun. 23, 2017.

(51) Int. Cl. A61F 2/14

(52) U.S. Cl. CPC A61F 2/148; A61F 2/142

(57) ABSTRACT

Devices and systems adapted for at least one of storing a corneal device and positioning a corneal device onto target tissue.

### CROSS REFERENCE TO RELATED APPLICATIONS

This application claims priority to U.S. Patent Application 62/524,201, filed Jun. 23, 2017, which is incorporated by reference herein. This application is related to U.S. Pat. No. 6,581,993 and WO2016/028275, which are incorporated by reference herein in their entireties.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Corrective eye procedures for improved vision may utilize a small corneal device, which can be too flexible and too small to be properly and reliably manipulated by hand, and which may also be difficult to manipulate with common tools such as tweezers or sinskey hooks.

Some existing system and methods require two hands for deploying a corneal device, with the second hand using a secondary tool to facilitate the deployment of the corneal device from a delivery tool. Mechanical forces used to manipulate the corneal device may damage the corneal device, so using a secondary tool to deploy a corneal device can have disadvantages.

Additionally, depending on the material of the corneal device, the corneal device may need to remain hydrated during storage. Preparing a delivery device and hydrated corneal device for use can cause the corneal device to become disassociated from the delivery device via fluid movement. For example, if parts of a packaging system near the corneal device are moved apart, the movement can cause fluid surface tension to pull the corneal device away from the delivery device, possibly causing loss of the corneal device.

Corneal devices that are hydrogel-based or have features that are small such as diameter, thickness, edge profile, etc. are hard to handle or manipulate by the surgeon or person performing the implantation. The device can become dislocated, bent, curled, scratched, or damaged in general without the use of a convenient well designed ergonomic delivery device.

Additional devices, systems, and methods are needed that can utilize surface tension of a fluid to safely, easily, quickly, and reliably perform at least one of the following: removing a corneal device from packaging and moving at least one support or storage device from an applicator member; and transferring a corneal device from an applicator member to corneal tissue.

Additional devices and system are needed that allow surface tension and capillary forces to retain a corneal device within a small area minimally affected by transportation forces through geometries that are less costly to form, to ensure the corneal device is presented in proper orientation.

### SUMMARY OF THE DISCLOSURE

One aspect of the disclosure is an elongate corneal device applicator, including a handle and an applicator surface adapted to interface with a surface of a corneal device, the applicator having a longitudinal axis extending along its length, the longitudinal axis passing through the applicator surface.

In a side view, an angle between the longitudinal axis and a plane or axis passing through at least a portion of the applicator surface, optionally at least the periphery of the applicator surface, is greater than 45 degrees, optionally greater than 55 degrees.

The longitudinal axis can pass through a central region of the applicator surface, even if the central region includes or is defined by an aperture in the applicator surface.

The applicator can further comprise a fluid channel extending proximally from an aperture in the applicator surface, the fluid channel being in fluid communication with the external environment that is external to the applicator at a location proximal to the applicator surface.

The applicator surface, even if curved, can have a diameter that is greater than a diameter of the corneal device, but optionally not greater than 2 times the diameter of the corneal device, and optionally not greater than 1.5 times the diameter of the corneal device.

The applicator surface diameter can be less than the outermost dimension (e.g., outer diameter) of the handle region.

The applicator can further include a cap interface that is adapted to interface with a cap and stabilize the relative positions of the applicator and cap. The cap interface can be proximal to the applicator surface.

One aspect of the disclosure is an elongate corneal device applicator, including a handle and an applicator surface adapted to interface with a surface of a corneal device, the applicator having a configuration of a marker, the marker not being a hockey stick configuration.

The applicator can further comprise a fluid channel extending proximally from an aperture in the applicator surface, the fluid channel being in fluid communication with the external environment that is external to the applicator at a location proximal to the applicator surface.

The applicator surface, even if curved, can have a diameter that is greater than a diameter of the corneal device, but optionally not greater than 2 times the diameter of the corneal device, and optionally not greater than 1.5 times the diameter of the corneal device.

The applicator surface diameter can be less than the outermost dimension (e.g., outer diameter) of the handle region.

The applicator can have a cap interface that is adapted to interface with a cap and stabilize the relative positions of the applicator and cap. The cap interface can be proximal to the applicator surface.

One aspect of the disclosure is an elongate corneal device applicator, including a handle and an applicator surface adapted to interface with a surface of a corneal device, the applicator having an elongate configuration other than an “L” configuration and other than a general hockey stick configuration.

One aspect of the disclosure is a cap for a corneal device apparatus, the cap sized and configured to be disposed over a distal end of a corneal device applicator.

The cap can be configured with at least one surface to form a distal end of a corneal device nest, the applicator forming a proximal end of the nest.

The cap can include a plurality of arms partially defining a corneal device nest, optionally wherein the plurality of arms are at least three arms, optionally at least four arms, optionally at least five arms, or optionally at least six arms. The plurality of arms can be disposed at a distal end of the cap. The plurality of arms can be coupled together at their distal ends. Each set of adjacent arms of the plurality of arms can define a plurality of spaces in between each set of adjacent arms. The spaces can be in fluid communication with an internal volume defined by the cap, the internal volume being in communication with a proximal end aperture adapted to receive therein a corneal device applicator. The plurality of arms can extend proximally from the distal end of the cap. The plurality of arms can extend radially outward from where they meet each other, then proximally toward a proximal end of the cap.

The cap can have an internal volume and a proximal end opening sized and configured to receive an applicator member.

The distal end of the cap can have a plurality of apertures therein so that the internal volume is in fluid communication with an external environment distal to the cap.

The cap, in a side view, can have a distal end, optionally defined by a plurality of arms, that is not orthogonal to a longitudinal axis of the cap.

The cap, in an end view of the cap, can have a plurality of apertures each extending through a distally facing surface of the cap, the aperture having general flower petal configurations with rounded outer edges.

The cap can include a distal end region that partially defines a corneal device nest, the distal end region having, in a side view, an outer dimension less than an outer dimension of a proximal end of the cap, optionally with a beveled surface somewhere in between.

One aspect of the disclosure is a corneal device storage and/or positioning system, comprising any of the applicator members described or claimed herein, and any of the caps described or claimed herein.

One aspect of the disclosure is a corneal device storage and/or positioning system, comprising: a corneal device applicator member having a marker configuration; and a cap sized and configured with an internal region or volume to fit completely over and around a distal end of the applicator member, the applicator member and the cap defining a corneal device nest for stabilizing a corneal device therein. The applicator member can be any of the applicator member claimed or described herein. The cap can be any of the caps claimed or described herein.

The disclosure also includes any method of using any of the applicators, caps, or systems herein, as part of a process to prepare for positioning or positioning a corneal device.

### BRIEF DESCRIPTION OF THE DRAWINGS

FIG. 1A is a perspective view of an exemplary assembled corneal device placing device in a closed configuration that may be used for storage/transportation.

FIG. 1B is an exploded view of the device shown in FIG. 1A, showing the components disassociated from one another.

FIG. 1C is a top view of the device of FIG. 1A.

FIG. 1D is a side view of the device of FIG. 1A.

FIG. 1E is a cross-sectional view of the device of FIG. 1D.

FIG. 1F is a side view of an exemplary corneal device.

FIG. 2A is a side view of a cap positioned over an application member.

FIG. 2B is a cross-sectional view of FIG. 2A.

FIG. 2C shows Detail A of FIG. 2B

FIG. 3A is a perspective view of an exemplary applicator member.

FIG. 3B is a cross-sectional view of FIG. 3A.

FIG. 3C shows detail B from FIG. 3B.

FIG. 4A shows a perspective front view of an exemplary cap.

FIG. 4B shows a side view of the cap from FIG. 4A.

FIG. 4C shows a front view of the cap from FIG. 4A.

FIG. 4D shows a back perspective view of the cap from FIG. 4A.

FIGS. 5A, 5B, and 5C illustrate an exemplary sequence of placing a corneal device on a surface using any of the applicator members herein. FIG. 5A is before the corneal device is placed, FIG. 5B shows placing the device on the surface, and FIG. 5C shows the applicator member lifted away from the surface, while the corneal device preferentially adheres to the surface, not the applicator member.

### DETAILED DESCRIPTION

The disclosure herein relates generally to devices and systems for at least one of storing a corneal device and positioning a corneal device onto target tissue. It should be noted that the disclosure herein describes the placement of a corneal device (e.g., inlay) as a mere example of how the placement devices can be used. In some embodiments, the device and systems can be used to place a corneal device onto corneal tissue, such as an inlay onto a stromal bed. The disclosure herein generally uses surface tension of a fluid along with pressure differentials to allow simple, reliable and accurate placement of a disk with one hand not otherwise attainable. The capillary and surface tension forces described herein can be used, however, to transport almost any object where precise and reliable placement is desired.

A merely exemplary advantage of devices herein is that the designs allow very minimal water to be introduced onto the corneal device during delivery of the device. This is advantageous when delivering transparent implants because transparent implants are difficult to see. This low water content ensures easy handling and manipulating by the surgeon during centration and desired movement of the implant into position.

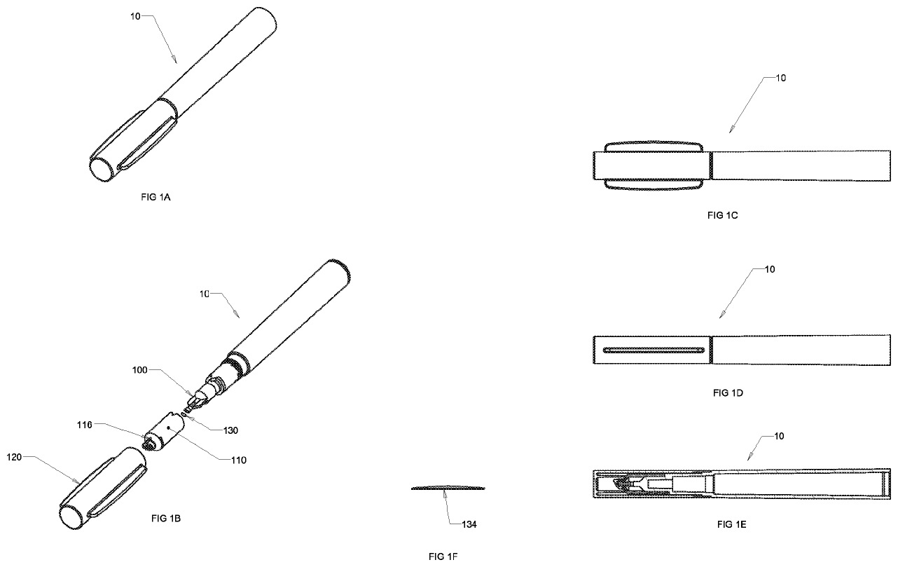

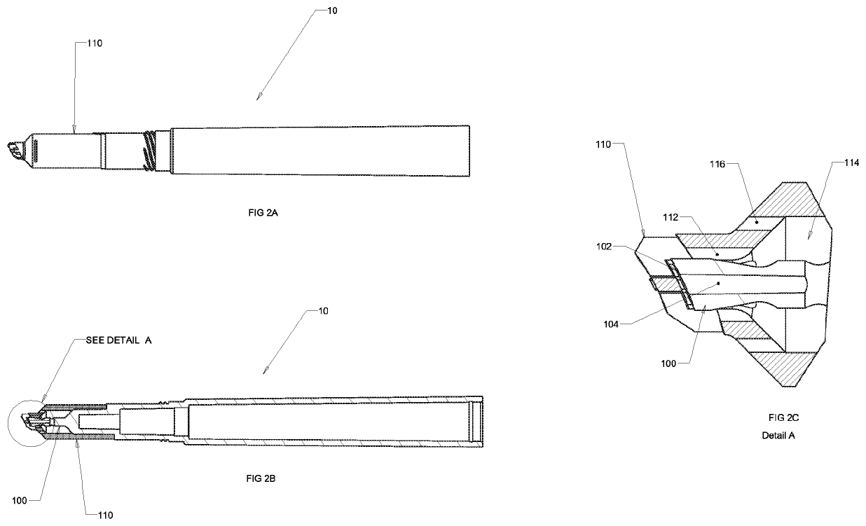

Referring generally to FIGS. 1A-1E, an exemplary assembled corneal device-placing system (10) comprises device applicator (100), a retaining cap (110), and an optional sealing/marker cap (120). The retaining cap may also be referred to as a support cap. In addition, device 10 is adapted and configured to be able to position corneal device 130 at a location in or on a cornea. Device 10 may be used to position corneal device 130 in combination with a liquid such as saline solution and a vapor such as air. It should be noted that at liquid-air interfaces, surface tension results from the greater attraction of liquid molecules to each other (due to cohesion) than to the molecules in the air (due to adhesion).

The systems and methods herein can be used with any suitable corneal device, such as any of the corneal devices described in US2011/0218623, which is fully incorporated by reference herein. For example, without limitation, the corneal device can have a diameter between 1-4 mm (e.g., 1-3 mm, or 2 mm), a thickness of 5 microns to 200 microns (e.g., 10 microns—150 microns, or 10 microns—50 microns), and made from a hydrogel material with a water content of at least 50%. Other corneal devices can, however, be stored and/or positioned using the device and/or systems herein.

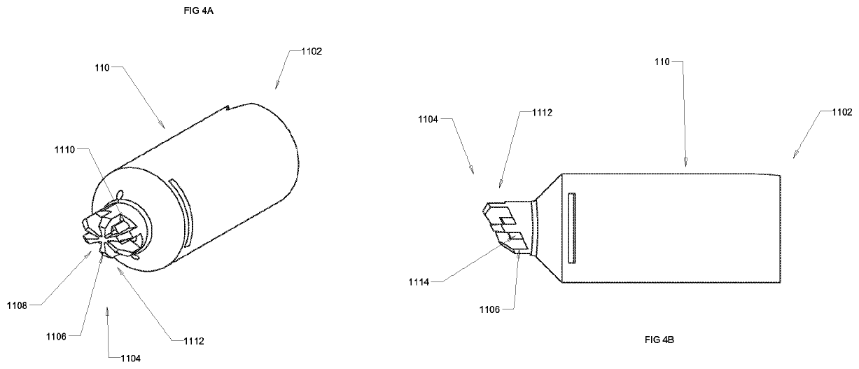

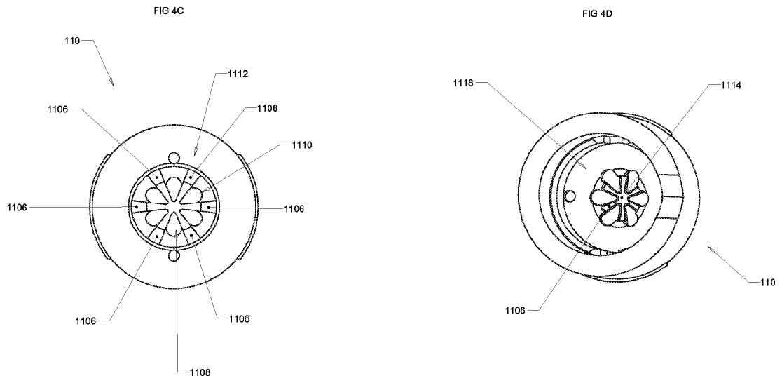

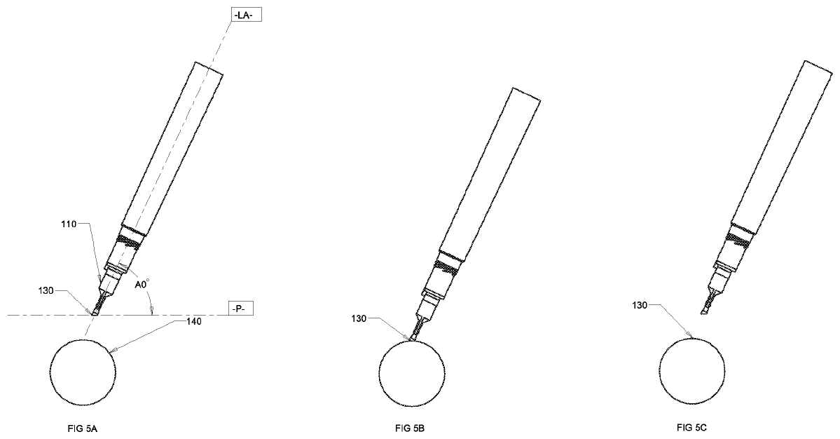

Referring to FIGS. 3C and 1F, device applicator (100) includes a retaining surface (102) and a capillary tube (104). The retaining surface (102) has a smaller total surface area than the final adhering surface of the corneal device (134) that adheres to corneal tissue. For example, in a merely exemplary embodiment, a 2 mm diameter corneal device can be held in place against surface 102 with a 2.1 mm diameter surface 102, less a 0.75 mm diameter capillary tube aperture, resulting in 3.14 mm² corneal device area versus a 3.02 mm² area for retaining surface 102. This allows for the ultimate transfer of the corneal device from the distal end of applicator 100 to the targeted deployment substrate 140 (e.g., corneal tissue) shown in FIGS. 4A-4C. Tube (104) acts as a capillary tube and helps retain the corneal device during deployment by surface tension of the fluid within tube 104. Tube 104 also serves as a conduit of vacuum pressure as described below. Channel 104 is in fluid communication with orthogonal channel 107 (see FIG. 3C), which passes through the outer surface of applicator 100. Channel 107 provides a pathway for channel 104 to be in fluidic communication with the ambient air proximal to where applicator corneal device 130 interacts with surface 102. The specific configuration of channel 107 is not critical, as long as it opens to the environment outside of applicator 100. The presence of channels 104 and 107 is, however, important for performance.

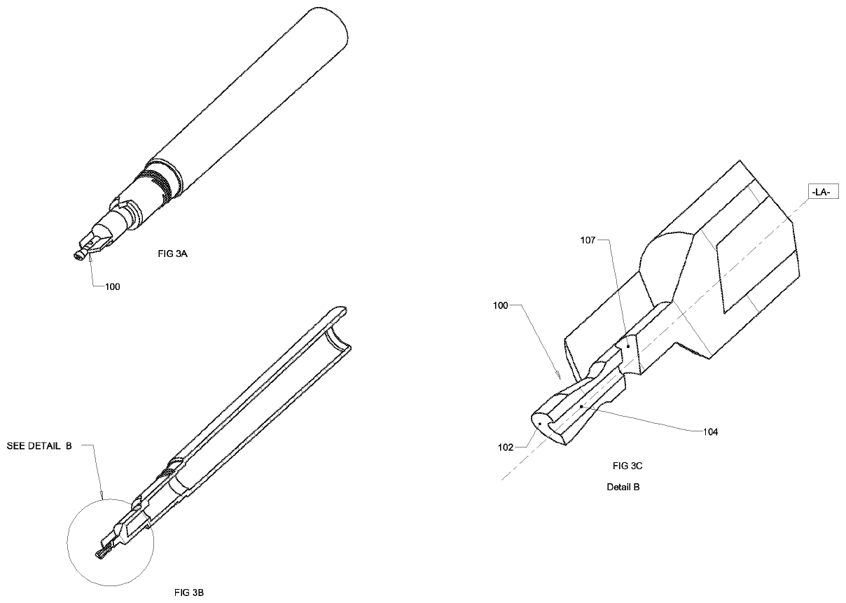

Referring to FIG. 3C (which provides a perspective sectional view of the distal region of applicator 100), the retaining cap (110) slides over the distal end of applicator (100) with a small amount of clearance 112 between the cap 110 and the applicator 100. As the retaining cap 110 is removed by moving it away from applicator 100, the volume contained between the tip of applicator 100 and the retaining cap (110) is increased, thereby lowering the pressure within the volume. This negative pressure is transferred to the top-portion (anterior side) of the corneal device through the capillary tube (104), which holds the corneal device to the placement tip (at surface 102) during the removal of the retaining cap 110. The small amount of clearance (112) allows the previously described negative pressure to equalize with the pressure outside of the retaining cap (110). This ensures the corneal device does not get pulled up into the capillary tube (104). Additional passageways (116), see FIG. 2C, may also be formed in the retaining cap (110) to allow for a quicker transfer of pressure.

FIGS. 4A-4D illustrate views of retaining (support) cap 110. FIG. 4A is a front perspective view, with proximal end 1102 and distal end 1104. The distal end of applicator 100 (not shown) is sized to be advanced within the opening in the proximal end 1102 in cap 110. The distal region of cap 110 includes a cage like region 1112, which includes a plurality of retaining members 1106 (in this embodiment there are six, but only 1 is labeled for clarity) that are coupled together at their distal ends. There is a space 1108 between each of the adjacent retaining members 1106. The spaces 1108 between retaining member 1106 are in fluid communication with the internal volume of cap 110. The side view of cap 110 shown in FIG. 4B illustrates an internal volume 1114 inside cap 110, which can be seen through the retaining member 1106. The retaining members 1106, from where they come together (they can be made integral, from the same starting material), extend radially outward and then proximally toward the proximal end of cap 110. The cap has a beveled region proximal to the distal cage where the outer surface bevels outward to the diameter of the proximal region. The distal region of cap 110 thus does not extend as far radially outward as the proximal end of cap 110. The retaining members and surface 102 of the applicator at least partially define the nest in which the corneal device is retained.

FIG. 4C is a front view of cap 110. The six equally spaced retaining members 1106 can be seen, as well as spaces 1108 (only 1 labeled for clarity) in between retaining members 1106. The cap cage region also includes a plurality of flower petal-shaped apertures 1110 (see the 1 aperture labeled in FIG. 4A; and also shown in FIG. 4D). As shown in FIG. 4C, the retaining members 1106 meet one another in a central region. The thickness of each retaining member 1106, moving in a radially outward direction, increases, which can be seen in FIG. 4C. In merely exemplary embodiments, the thickness of each retaining member is 0.2-0.4 mm (e.g., 0.3 mm) at its most radially inward location, 0.4-0.6 mm (e.g., 0.5 mm) at the inner wall, and 0.6-0.8 mm (e.g., 0.7 mm) at the outer wall. Exemplary thicknesses for the retaining members 1106, measured from the concave distal surface to the convex proximal surface, are 0.8-1.2 mm (e.g., 1 mm). One goal when choosing dimensions and configurations of the retaining members may be to maximize fluid drainage to possibly eliminate the need for a wicking step, while minimizing the likelihood the corneal device escaping through spaces 1108.

FIG. 4D shows a perspective view of the proximal end of cap 110—the end into which the distal tip of applicator 100 is positioned when cap 110 is placed over the distal end of applicator 100. Cap 110 has a general internal volume 1118 defined by the inner surfaces of cap 110, including the inner surfaces of retaining members 1106 (only one of the retaining members 1106 is labeled). The general internal volume 1118 of cap 110 includes, when applicator 100 is fully positioned into cap 110, corneal device nest 1114 at least partially defined by surface 102 and the inner surfaces of retaining members 1106.

The curvature of applicator surface 102 is as close as possible to the curvature of the anterior surface of corneal device 130 (even though there is an aperture in surface 102). The applicator surface 102, when manufactured, can thus be configured to correspond as closely as possible to whatever the curvature is of the intended corneal device 130. In some embodiments the surface 102 has a spherical curvature (excluding the aperture in the central region of surface 102) that corresponds to a spherical anterior surface of a corneal device. The inner surfaces of retaining members 1116 defining the distal end of nest 1114 can similarly have a curved configuration that matches the curvature of a proximal surface of the corneal device. Even though the retaining members 1106 have apertures between them, the inner surfaces of retaining members 1116 can still be considered to have a curvature based on a curve that may be imparted to the distal end of the corneal device nest before further apertures are machined into the cap 110. So even though the distal end of the nest may not define a continuous surface (it has apertures), it can still be considered to have a curvature. Similarly, the anterior end of the nest has a curvature even though an aperture extends therethrough.

The cage region of cap 110 has, in this embodiment, six apertures between retaining members 1106. Those apertures are in fact defined by the surfaces of retaining members 1106. The retaining members are merged together at a central location of the nest, which helps retain the corneal device in the device nest. The apertures are sized to prevent the corneal device from escaping out of the nest through any of the apertures.

In some exemplary embodiments, the system parts are configured and sized such that surface 102 is 100 microns (+/−50 microns) away from the inner surface of retaining members 1106. The distance between surfaces may be chosen based on the thickness of the corneal device.

As discussed above, when cap 110 is removed (pulled off) applicator 100, the corneal device remains adhered to distal tip surface 102 of applicator 100. This is considered to be a preferential adhesion to applicator 100 rather than cap 110. Stated alternatively, the apparatus 10 is adapted and configured so that corneal device 130 will preferentially adhere to distal tip surface 102 of applicator 100, not the cap 110, when cap 110 is pulled off applicator 100 (or applicator 100 is pulled from cap 110. The apertures 1110 reduce the surface area of the distal region of nest adjacent the corneal device 130, which reduces the amount of contact the distal region of the nest (i.e., the cap) has with the corneal device 130. Having a system 10 in which corneal device 130 has less contact with cap 110 than applicator 100 helps the corneal device 130 preferentially adhere to applicator tip surface 102 when cap 110 is removed from the applicator 100. The concept of preferential adhesion is described more fully in WO2016/028275, the description of which is incorporated by reference herein.

It was observed through testing that prior to the removal of retaining cap 110, fluid did not need to be removed, or wicked away, in order for corneal device (130) to remain on the distal tip of applicator (100) after retaining cap 110 was pulled away. This eliminates an additional wicking step during the placement procedure, thereby making the overall process more efficient. Referring to the exemplary method of placement shown in the sequential FIGS. 4A-4C, as the corneal device is positioned over the target deployment substrate (140—e.g. corneal tissue), the corneal device remains on the applicator tip through surface tension of the fluid between the corneal device (130) and the retaining surface (102) and capillary forces through the capillary tube (104). As the corneal device comes in contact with the target deployment surface (140), the larger surface area of the final adhering surface of the disk 134 (in this case the posterior surface of the corneal device), compared to the smaller surface area of the tip retaining surface (102), causes the corneal device to transfer (be displaced) from surface 102 and onto the target deployment 140. This breaks the surface tension of the capillary fluid allowing for a small volume of fluid to transfer to the anterior side of the disk (130).

Referring to FIGS. 1A-1E, and the detailed sectional view of FIG. 2C, in the transportation configuration (cap 110 advanced as far proximally as possible over applicator 100 distal end), the retaining cap 110 contains the corneal device laterally in a tight confine. This is achieved through walls that lead up (proximally) and around the applicator 100 placement tip, and by the configuration of the placement tip itself. In addition, the placement tip of applicator 100 and retaining cap 110 surfaces adjacent the corneal device may be shaped (i.e., configured) to match the contours of the anterior and posterior surfaces of the corneal device. For example, a corneal inlay or onlay may be concave on the top/anterior surface and convex on the bottom/posterior surface. By configuring the surfaces adjacent the corneal device to follow the surface shapes, fluid volumes between the corneal device and the adjacent surfaces of device 10 are kept equal (or substantially equal),thereby reducing differential forces affecting the position of the corneal device during vibration and sudden physical shock. These features help keep the flexible corneal device in position and orientation during vibration and impact forces that typically occur during transportation. This helps ensure more accurate presentation and placement of the corneal device onto the target location.

Some earlier applicator devices include a distal bend, such that the device approximates the configuration of a standard hockey stick (the lower portion and handle defining an angle that is 90 degrees or more). The applicators herein do not have this general hockey stick configuration, but rather have a “marker” configuration. That is, the applicators herein are elongate and do not have a distal bend near the distal end. This allows the applicator members to be held and used in virtually the same way as one would contact a marker with a piece of paper. Another way of describing the shape is that the applicator member, which includes a handle region and applicator surface, has a longitudinal axis (see axis LA in FIG. 3C) that extends through the handle region and also passes through the applicator surface 102 to which the corneal device will be adhered to at some time when removing the support device (in this case the cap 110). In this particular embodiment, and as shown in FIG. 5A, a plane or axis (see “P” in FIG. 5A) passing through at least a portion of the distal tip of application 110 and the longitudinal axis LA of the applicator member 110 define an angle “A” that is 45 degrees or greater. In this embodiment the angle “A” shown in FIG. 5A is greater than 45 degrees. Plane

P exists even though surface 102 has a curved configuration. Plane P is essentially passing through the outer periphery of surface 102, even if plane P does not pass through the entire surface 102 (since surface 102 is curved). Cap 110 is sized and configured to be positioned around the distal end of applicator 110.

The system may also include a marker (which may be sealing) cap, such as cap 120 shown in FIGS. 1A-1E. A marker cap such as cap 120 may help in keeping cap 110 stay secured to applicator member 100, and it can also be used to maintain saline in the nest environment so that the corneal device stays hydrated. The fitting between cap 120 and the rest of the system can be a secure and sterile seal. Optionally, the entire system 10 can be inside an additional sterile packaging. Cap 120 can optionally have one or more holes in it if the entire system is disposed in another sterile packaging.

Through at least one or more of unequal surface areas (described above), capillary tube (described above) and the natural change of volume leading to negative pressures (described above), the devices herein provide for the quick and reliable transfer of a corneal device with fewer required steps and lower manufacturing costs.

Any of the systems and applicator members described herein, such as shown in FIGS. 1A-1E, 2A, 2B, 3A-3C, or 5A-5C, may be the subject of a design patent application that is filed as a continuing application based on this disclosure. For example, the disclosure herein, including FIGS. 1A, 1C, 1D, adequately describes any ornamental feature of the assembled system. It is understood that alternative to, for example, FIGS. 1A, 1C, 1D, are included herein (even without being specifically shown), the alternatives including optional hashed lines on any of the lines shown in FIGS. 1A, 1C, and 1D. It is similarly understood that alternatives to figures of applicator members are included herein (even without being explicitly shown), the alternatives includes hashed lines on any of the lines shown in the figures that include applicator members.

1. An elongate corneal device applicator, including a handle and an applicator surface adapted to interface with a surface of a corneal device, the applicator having a longitudinal axis extending along its length, the longitudinal axis passing through the applicator surface.

2. The applicator of claim 1, wherein, in a side view of the applicator, an angle between the longitudinal axis and a plane or axis passing through at least a portion of the applicator surface, optionally at least the periphery of the applicator surface, is greater than 45 degrees, optionally greater than 55 degrees.

3. The applicator of claim 1, wherein the longitudinal axis passes through a central region of the applicator surface, even if the central region is defined by an aperture in the applicator surface.

4. The applicator of claim 1, wherein the longitudinal axis passes through a central aperture defined by the applicator surface.

5. The applicator of any of claim 1, further comprising a fluid channel extending proximally from an aperture in the applicator surface, the fluid channel being in fluid communication with the external environment that is external to the applicator at a location proximal to the applicator surface.

6. The applicator of claim 1, wherein the applicator surface, even if curved, has a diameter that is greater than a diameter of the corneal device, but optionally not greater than 2 times the diameter of the corneal device, and optionally not greater than 1.5 times the diameter of the corneal device.

7. The applicator of claim 1, wherein the applicator surface diameter is less than the outermost dimension (e.g., outer diameter) of the handle region.

8. The applicator of claim 1, wherein the applicator further comprises a cap interface that is adapted to interface with a cap to stabilize the relative positions of the applicator and the cap.

9. The applicator of claim 8, wherein the cap interface is proximal to the applicator surface.

10. An elongate corneal device applicator, including a handle and an applicator surface adapted to interface with a surface of a corneal device, the applicator having a configuration of a marker, the marker not being a hockey stick configuration.

11. An applicator of claim 10, wherein the marker configuration does not have a hockey stick configuration that includes a bend closer to a distal end of the applicator than a proximal end of the applicator.

12. The applicator of claim 10, further comprising a fluid channel extending proximally from an aperture in the applicator surface, the fluid channel being in fluid communication with the external environment that is external to the applicator at a location proximal to the applicator surface.

13. The applicator of claim 10, wherein the applicator surface, even if curved, has a diameter that is greater than a diameter of the corneal device, but optionally not greater than 2 times the diameter of the corneal device, and optionally not greater than 1.5 times the diameter of the corneal device.

14. The applicator of claim 10, wherein the applicator surface diameter is less than the outermost dimension (e.g., outer diameter) of the handle region.

15. The applicator of claim 10, wherein the applicator includes a cap interface that is adapted to interface with a cap and stabilize the relative positions of the applicator and the cap.

16. The applicator of claim 15, wherein the cap interface is proximal to the applicator surface.

17. An elongate corneal device applicator, including a handle and an applicator surface adapted to interface with a surface of a corneal device, the applicator having an elongate configuration other than an “L” configuration and other than a general hockey stick configuration.

18. An applicator of claim 17, wherein elongate configuration does not have a configuration that includes a bend closer to a distal end of the applicator than a proximal end of the applicator.

19. A cap for a corneal device apparatus, the cap sized and configured to be disposed over a distal end of a corneal device applicator.

20. The cap of claim 19, wherein the cap is configured with at least one surface to form a distal end of a corneal device nest, the applicator forming a proximal end of the nest.

21. The cap of claim 19, wherein the cap includes a plurality of arms partially defining a corneal device nest, optionally wherein the plurality of arms are at least three arms, optionally at least four arms, optionally at least five arms, or optionally at least six arms.

22. The cap of claim 21, wherein the plurality of arms are disposed at a distal end of the cap.

23. The cap of claim 21, wherein the plurality of arms are coupled together at their distal ends.

24. The cap of claim 21, wherein each set of adjacent arms of the plurality of arms defines a plurality of spaces in between each set of adjacent arms.

25. The cap of claim 24 wherein the spaces are in fluid communication with an internal volume defined by the cap, the internal volume in communication with a proximal end aperture adapted to receive therein a corneal device applicator.

26. The cap of claim 21, wherein the plurality of arms extend proximally from the distal end of the cap.

27. The cap of claim 21, wherein the plurality of arms extend radially outward from where they meet each other, then proximally toward a proximal end of the cap.

28. The cap of claim 21, wherein the cap has an internal volume and a proximal end opening sized and configured to receive an applicator member.

29. The cap of claim 28, wherein the distal end of the cap has a plurality of apertures therein so that the internal volume is in fluid communication with an external environment distal to the cap.

30. The cap of claim 21, wherein the cap, in a side view, has a distal end, optionally defined by a plurality of arms, that is not orthogonal to a longitudinal axis of the cap.

31. The cap of claim 21, wherein the cap, in an end view of the cap, has a plurality of apertures each extending through a distally facing surface of the cap, the aperture having general flower petal configurations with rounded outer edges.

32. The cap of claim 21, wherein the cap includes a distal end region that partially defines a corneal device nest, the distal end region having, in a side view, an outer dimension less than an outer dimension of a proximal end of the cap, optionally with a beveled surface somewhere in between.

33. A corneal device storage and positioning system, comprising any of the applicator members described or claimed herein, and any of the caps described or claimed herein.

34. A corneal device storage and positioning system, comprising:
a corneal device applicator member having a marker configuration; and
a cap sized and configured with an internal region or volume to fit completely over and around a distal end of the applicator member, the applicator member and the cap defining a corneal device nest for stabilizing a corneal device therein.
